(19) United States
(12) Patent Application Publication
(10) Pub. No.: US 2021/0338141 A1
(43) Pub. Date: Nov. 4, 2021

(54) SEIZURE DETECTION SYSTEM IN MOBILE SUBJECTS

(71) Applicant: KORWAVE, LLC, Surprise, AZ (US)

(72) Inventor: Patrick S. McFARLAND, Surprise, AZ (US)

(21) Appl. No.: 17/273,477

(22) PCT Filed: Sep. 5, 2019
(86) PCT No.: PCT/US2019/049777

§ 371 Date: Mar. 4, 2021

Related U.S. Application Data
(60) Provisional application No. 62/728,568, filed on Sep. 7, 2018.

(51) Int. Cl. A61B 5/00; C08L 5/06; C08L 89/06; C08J 3/22; C08J 3/075; H01B 1/24

(52) U.S. Cl. CPC A61B 5/4094; C08L 2205/03; A61B 5/0006; A61B 5/0022; A61B 5/7264; C08L 5/06; C08L 89/06; C08J 3/226; C08J 3/075; H01B 1/24; C08L 2201/52; C08L 2203/20; C08L 2203/02; C08L 2310/00; A61B 5/746

(57) ABSTRACT

An electrode for use in ambulatory BEG signal collection includes an electrode contact comprising a gelatin or hydrogel polymer having low input impedance. The electrode contact retains adhesive ability and low input impedance over long periods of time. A system using the electrode includes circuits and software for analyzing EEG signals to detect and report ictal events in real time. At least part of the system is wearable; and advantageously part of the system resides in a cloud based system. A signal analyzer incorporated in the system provides real time ictal signal identification through physical graphing and cross-correlation. In some aspects the analyzer also provides confirmation of ictal signal identification by physical graphing and machine deep learning. Analysis of physical graphs may conveniently be performed using a graphics processing unit to speed processing.

### CROSS-REFERENCES TO RELATED APPLICATIONS

This application claims priority from U.S. Provisional Patent Application 62/728,568, filed Sep. 7, 2018, the entire contents of which are incorporated herein by reference.

### FIELD OF THE INVENTION

The present invention relates to seizure detection in mobile subjects.

### BACKGROUND OF THE INVENTION

### Epileptic Seizure

Epileptic seizures affect millions of children and adults worldwide; an estimated 1% of people in the United States suffer from epileptic seizures. Epilepsy is not limited to humans. For example, epilepsy is fairly common in several breeds of dogs. Seizures may occur unpredictably, apparently randomly, rendering the epileptic subject incapacitated. This unpredictability is a significant source of concern and anxiety in epileptic subjects, their relatives and caregivers. Rapid, real-time detection of epileptic seizures in ambulatory subjects, and alerting of responsible persons (parents, custodians, and care givers of human subjects; owners of pets), would go a long way toward alleviating these concerns and anxieties, but have heretofore remained unachieved, at least in part because of difficulties in collecting brain signals in ambulatory subjects, and at least in part because of difficulties presented by analysis and evaluation of large sets of brain signal data required to discriminate ictal (seizure) from interictal (non-seizure) brain activity.

Electroencephalography in the clinic, i.e., in relatively immobile subjects, is relatively facile and well-developed. A conductive adhesive (cement) is used to attach conductive electrodes to the scalp of a subject to be studied. Brain waves are collected through the electrodes and conducted to an EEG instrument, where they are filtered, amplified, and presented to the clinician as graphs of multiple brain wave spectral bands. The clinician reads these graphical images and identifies signals indicative of potential ictal events. The clinician may confirm suspected ictal signals, e.g., by viewing video of the subject to rule out suspected signals that coincide with (and are probably caused by) patient movement, e.g. mouth or tongue motion, kinetic motor function, sneezing, etc.

EEG detection periods in the clinic are generally short-6 to 24 hours—and thus the quantity of encephalographic and video data collected is not generally intractable for a well-trained clinician. Of course, such detection and confirmation is, of necessity, post hoc, as the clinician is rarely present when an ictal event occurs. Only if the clinician happens to be present at the time the EEG is collected, and confirmation through secondary means is accomplished in real time (e.g., by direct observation of the subject by the clinician), is it possible to detect and confirm an ictal event in real time. As it is impractical to have a clinician follow a subject in day-to-day life, whether in person or remotely, it is impractical for clinicians to detect seizures by EEG in real-world environments with current technology. Thus, it would be desirable to be able to detect and confirm the occurrence of ictal events in real time, without the consistent monitoring of a trained clinician.

### Electroencephalography in Ambulatory Subjects

Electrodes

Whatever the challenges of detecting suspected ictal events using EEG in the clinic, they are orders of magnitude greater in ambulatory subjects. EEG seizure detection and confirmation in ambulatory subjects are frustrated by several considerations, at least some of which are related to the electrodes used to collect brain signals. Electrodes used in mobile subjects must be suitable for long term use. They need to have low input impedance both initially, when first attached, and over a long term; they also must remain attached to the subject under kinetic circumstances (e.g., the subject's walking, running, jumping, sitting, standing, etc.). As a practical matter, they should ideally require little or no depilation, as such would be considered esthetically undesirable in humans, and somewhat impractical in pets, such as canines. Heretofore there have been no electrodes available for use in EEGs in a kinetic environment that have the combined properties of low input impedance in the long term and resilient ability to bind to, and retain electrical contact with, a subject's scalp in an ambulatory (non-clinical) environment. Nor have any electrodes been described that combine durable low input impedance, and adhesiveness, and that also require little or no depilation.

Data Analysis

Real time identification of ictal EEG signals in a non-clinical, ambulatory setting, is also frustrated by the difficulty of dealing with large data sets over time. Even in a subject that experiences a relatively large number of seizures in a given amount of time, the vast majority of EEG signal will be interictal (non-seizure). Moreover, a large amount of data that may at first blush appear to be ictal in nature, may turn out to be noise caused by normal motor function of the subject's body, such as facial and eye movement, coughing, sneezing, etc. While there have been algorithms suggested for collection of large sets of EEG data, and for identifying those signals that appear to be ictal in nature, the problem of doing so in real time, so that a clinician, parent, custodian, or pet owner may be promptly alerted of a seizure, has remained elusive. What is needed is a system, comprised of devices and algorithms running on those devices, that can handle large data sets in real time, detect a suspected ictal event, alert a responsible person(s) of suspected seizure onset, quickly confirm (or refute) ictal activity, and inform the responsible person(s) of this confirmation or refutation. There is also a need for a computer system capable of learning to distinguish between ictal and interictal events, thereby increasing the ratio of true-positive to false-positive indications of ictal events.

### SUMMARY OF THE INVENTION

The foregoing, and other related, problems are addressed by aspects and embodiments of the invention described herein.

In some embodiments, there is provided an electrode comprising an electrode composition and a conductive element. The electrode composition possesses both conductive and adhesive properties, such that when the electrode is placed on a subject's scalp, the electrode has low input impedance and maintains electrical contact with the subject's scalp, despite normal kinetic activity of the subject. In some embodiments, the electrode composition retains its adhesive nature over a long period of time and retains this adhesiveness, such that electrical contact is made with the subject's scalp, under kinetic conditions, e.g., when the subject is running, jumping, sitting, standing, sleeping, etc. The electrode also has an initial low input impedance. The input impedance of the electrode remains low over long term use, e.g., over periods of several days, weeks, or even months. In some embodiments, the electrode composition comprises: (a) a polysaccharide gelling agent; (b) a low molecular weight carbohydrate; (c) a polypeptide gelling agent; (d) a polymeric gel thickener; (e) an emulsifier; (f) a conductive carbon species; and (g) water. In some embodiments, the composition also includes one or more buffer species to maintain the pH of the composition within workable limits, e.g. pH 2 to 6, more specifically pH 2 to 4 or pH 2.9 to 3.3. In some embodiments, the buffer species is citric acid, calcium citrate, or a mixture of citric acid and calcium citrate. In some embodiments, the polysaccharide gelling agent is Methoxyl Pectin. In some embodiments, the Methoxyl Pectin is High Methoxyl Pectin. In some embodiments, the polypeptide gelling agent is gelatin. In some embodiments, the polymeric gel thickener is carboxymethyl cellulose. In some embodiments, the emulsifier is polyvinylpyrrolidone. In some embodiments, the conductive carbon species is acetylene black.

In some embodiments, there is provided an electrode comprising an electrode composition and a conductive element. The electrode composition possesses both conductive and adhesive properties, such that when the electrode is placed on a subject's scalp, the electrode has low input impedance and maintains electrical contact with the subject's scalp, despite normal kinetic activity of the subject. In some embodiments, the electrode composition retains its adhesive nature over a long period of time and retains this adhesiveness under kinetic conditions, e.g., when the subject is running, jumping, sitting, standing, sleeping, etc. The electrode also has an initial low input impedance. The input impedance of the electrode remains low over long term use, e.g., over periods of several days, weeks, or even months. In some embodiments, the electrode composition comprises: (a) 1-5% (mass fraction) Methoxyl Pectin; (b) 30-60% (mass fraction) low molecular weight carbohydrate; (c) 1-10% (mass fraction) gelatin; (d) 1-10% (mass fraction) carboxymethyl cellulose; (e) 0.05-0.2% (mass fraction) acetylene black; (f) 0.3-1.0% (mass fraction) polyvinylpyrrolidone; and (g) q.s. water. In some embodiments, the composition further comprises a buffer species. In some embodiments, the buffer species is an acid, a base, or a combination of both an acid and a base. In some embodiments, the buffer species is citric acid, calcium citrate, or both. In some embodiments, the composition has a pH in a range of 2.0 to 4.0. In some embodiments, the Methoxyl Pecitin is Low Methoxyl Pectin. In some embodiments, the composition comprises 2.2% to 2.6% (mass fraction) of High Methoxyl Pectin. In some embodiments, the composition comprises 40-55% (mass fraction) low molecular weight carbohydrate. In some embodiments, the composition comprises 45-50% (mass fraction) low molecular weight carbohydrate. In some embodiments, the low molecular weight carbohydrate is sucrose. In some embodiments, the composition comprises 4-6% (mass fraction) gelatin. In some embodiments, the composition comprises 4-6% (mass fraction) carboxymethyl cellulose. In some embodiments, the composition comprises 0.075-0.125% (mass fraction) acetylene black. In some embodiments, the composition comprises 0.5-0.9% (mass fraction) polyvinylpyrrolidone. In some embodiments, the composition has a pH in a range of 2.5 to 3.5. In some embodiments, the composition comprises: (a) methoxyl pectin—2.3% to 2.5% (mass fraction); (b) sucrose—48.0% to 48.5% (mass fraction); (c) gelatin—4.7% to 4.9% % (mass fraction); (d) carboxymethyl cellulose—4.7% to 4.9% (mass fraction); (e) acetylene black—0.09% to 0.10% (mass fraction); (f) polyvinylpyrrolidone—0.6% to 0.7% (mass fraction); (g) a suitable buffer; and (h) q.s. water. In some embodiments, the methoxyl pectin is high methoxyl pectin. In some embodiments, the composition comprises: (a) high methoxyl pectin—2.4% (mass fraction); (b) sucrose—48.4% (mass fraction); (c) gelatin—4.8% (mass fraction); (d) carboxymethyl cellulose—4.8% (mass fraction); (e) acetylene black—0.1% (mass fraction); (f) polyvinylpyrrolidone—0.7% (mass fraction); (g) a suitable buffer; and (h) q.s. water. In some embodiments, the composition comprises: (a) high methoxyl pectin—2.42% (mass fraction); (b) sucrose—48.4% (mass fraction); (c) gelatin—4.84% (mass fraction); (d) carboxymethyl cellulose—4.84% (mass fraction); (e) acetylene black—0.097% (mass fraction); (f) polyvinylpyrrolidone—0.677% (mass fraction); (g) a suitable buffer; (h) q.s. water. In some embodiments, the composition has a pH in a range of 2.9 to 3.3. In some embodiments, the composition comprises: (a) high methoxyl pectin—2.4178% (mass fraction); (b) sucrose—48.356% (mass fraction); (c) gelatin—4.8356% (mass fraction); (d) carboxymethyl cellulose—4.8356% (mass fraction); (e) acetylene black—0.0967% (mass fraction); (f) polyvinylpyrrolidone—0.6769% (mass fraction); (g) a suitable buffer; (h) q.s. water. In some embodiments, the composition has a pH in a range of 2.9 to 3.3.

The invention also contemplates a method of making an electrode composition as disclosed herein, the process comprising: (a) placing and mixing the polypeptide gelling agent, the polymeric gel thickener, and optionally a buffer powder in a first container; (b) charging the water, the emulsifier, and the conductive carbon species powder into a second container; (c) thoroughly dispersing the conductive carbon species powder in the water to form a dispersion; (d) heating the dispersion in the second container to about 60° C.; (e) introducing the polysaccharide gelling agent into the second container and stirring to dissolve the; (f) polysaccharide; (g) charging the mixed polypeptide gelling agent, polymeric gel thickener, and buffer powder from the first container into the second container and stirring until the charged powder is dissolved; (h) charging the low molecular weight carbohydrate into the second container and stirring until the low molecular weight carbohydrate is dissolved; (i) discontinuing heating of the dispersion in the second container and stirring until the dispersion begins to thicken; and (j) allowing the dispersion to set to form the electrode composition. In some embodiments, the polysaccharide gelling agent is Methoxyl Pectin. In some embodiments, the Methoxyl Pectin is High Methoxyl Pectin. In some embodiments, the polypeptide gelling agent is gelatin. In some embodiments, the polymeric gel thickener is carboxymethyl cellulose. In some embodiments, the emulsifier is polyvinylpyrrolidone. In some embodiments, the conductive carbon species is acetylene black. In some embodiments, the composition comprising a buffer species. In some embodiments, the buffer species comprises citric acid, calcium citrate, or both. In some embodiments, the composition has a pH of from about 2 to about 4.

The invention also contemplates a method of making an electrode composition as disclosed herein, the process comprising: (a) placing and mixing the gelatin, the carboxymethyl cellulose, and buffer powders in a first container; (b) charging the water, the polyvinylpyrrolidone, and the acetylene black powders into a second container; (c) thoroughly dispersing the acetylene black in the water to form a dispersion; (d) heating the dispersion in the second container to about 60° C.; (e) introducing the methoxyl pectin into the second container and stirring to dissolve the methoxyl pectin; (f) charging the mixed gelatin, carboxymethyl cellulose, and buffer powders from the first container into the second container and stirring until the powders are dissolved; (g) charging the low molecular weight carbohydrate into the second container and stirring until the low molecular weight carbohydrate is dissolved; (h) discontinuing heating of the dispersion in the second container and stirring until the dispersion begins to thicken; and (i) allowing the dispersion to set to form the electrode composition.

The invention also contemplates a method of making an electrode, comprising contacting an electrode composition as described herein with an electrically conductive element.

An alternate embodiment of the invention provides an electrode comprising a hydrogel polymer composition that is adhesive over a long period of time and retains this adhesiveness under kinetic conditions, e.g., when the subject is running, jumping, sitting, standing, sleeping, etc. The electrode also has an initial low input impedance. The input impedance of the electrode remains low over long term use, e.g., over periods of several days, weeks, or even months. In some aspects of the invention, the hydrogel comprises a co-polymer of poly(propylene glycol) diacrylate and pentaerythritol tetrakis(3-mercaptopropionate) monomer. The hydrogel is very hygroscopic, capable of absorbing aqueous solutions, such as buffered solutions. In some aspects of the invention, the aqueous solution is a physiologically acceptable aqueous buffer having a pH of about 7 to about 10. In some aspects, the poly(propylene glycol) diacrylate has an average monomer number (Mn) of about 800. In some aspects, the pentaerythritol tetrakis(3-mercaptopropionate) is about 95% pure. In some aspects, the physiologically acceptable aqueous buffer is an aqueous phosphate buffer or an aqueous bicarbonate-carbonate buffer. In some aspects, the electrode has an initial impedance of less than about 100 KΩ. In some aspects, the electrode, after prolonged usage, has an impedance of less than about 500 KΩ.

Aspects of the invention further provide a system for detecting a seizure comprising: An EEG signal collector comprising an analog front end having a power supply, said analog front end: providing a bias signal through a bias electrode; receiving an analog brain signal through at least two scalp electrodes; performing analog to digital conversion to prepare a digital signal representative of the analog brain signal; and passing the digital signal to a digital interface; the digital interface providing an interface between said analog front end and signal analyzer; the signal analyzer performing at least the following steps: sectioning the digital signal into time segments; transforming the time segments into at least one domain other than the time domain to form at least one transform; preparing at least one graph of said at least one transform; carrying out at least one of cross correlation or machine deep learning on said at least one graph to detect an ictal event; and, on detection of an ictal event, passing a signal to an alert subsystem; the alert subsystem adapted to provide an alert signal to a device adapted to receive the alert signal and present it to a subject or a responsible person.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1 is a depiction of an electrode according to the present invention.

FIG. 2 is a schematic diagram of a seizure detection and alerting system according to the present invention.

FIG. 3 is a block diagram of an algorithm according to the present invention that processes incoming digital signals representative of an EEG, detects potential ictal events, confirms or refutes potential ictal events, and presents an alert of a seizure to a responsible person in case an ictal event is confirmed.

FIG. 4 is a block diagram of an algorithm for collecting and processing EEG signals, detecting ictal events, and alerting caretakers of such ictal events, according to some embodiments the present invention.

FIG. 5. is a hardware block diagram showing a system for collecting and processing EEG signals, detecting ictal events, and alerting caretakers of such ictal events, according to some embodiments of the present invention.

### DETAILED DESCRIPTION OF THE INVENTION

The present invention provides devices, systems, and methods for acquiring electroencephalographic signals from a scalp of a subject, converting those signals to digital signals, analyzing those signals to discriminate ictal signals from interictal signals, and alerting responsible persons to the presence of seizures.

As used herein, the term “subject,” means an animal or human subject that has experienced, or is suspected of having experienced, at least one ictal event. A subject may or may not have been diagnosed with epilepsy or other seizure disorder. An exemplary animal subject would include a dog (canine), cat (feline), cow (bovine), pig (porcine), horse (equine), or other domesticated animal. Included within the scope of the term “subject” is the term “patient.”

The term “ictal” refers to seizure.

The term “caretaker” as used herein and in the claims includes both professional and non-professional humans charged with caring for the subject. In the case of human subjects, this may include physicians, nurses, and other healthcare professionals, as well as family members and others responsible for administering anti-seizure therapy to the subject. In the case of animal subjects, the term “caretaker” may include veterinarians, veterinary assistants, and other veterinary healthcare professionals, as well as humans charged with caring for the animal subject.

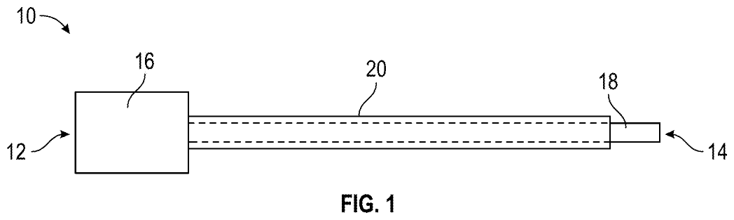

In one aspect of the invention, there is presented an electrode adapted for long-term use in a kinetic use environment, such as on an ambulatory human or animal. In some embodiments, the electrode is adapted for long-term attachment to a scalp of a human or animal. Turning to FIG. 1, an electrode 10 possesses distal 12 and proximal 14 ends and an electrical conductor 18. At the distal end 12 of electrode there is formed an electrode contact 16. The figures are not to scale—the electrode contact 16, as well as other features of the figures, may be of any suitable size and proportion. The electrode may further comprise shielding 20, which is an insulated conductor enclosed by a common conductive layer (i.e., the outer conductor is faraday shield of inner conductor carrying signal and there is an insulator between the two conductors) over the conductive element 18.

The electrode contact 12 is of such surface area and conductivity that the electrode as a whole has a suitable impedance, not only initially, but over long-term use in a kinetic usage environment, such as a mobile subject engaged in normal daily activities. High input impedance would lead to low signal current through the electrode, which would result in a low signal to noise ratio. Thus, it is important that the electrode contact 12 have low input impedance in order to maintain a high signal to noise ratio. In some embodiments, the suitable operating impedance of the electrode contact 12 is less than 500 KΩ, preferably less than 250 KΩ, more preferably less than 125 KΩ, and even more preferably less than 75 KΩ. In some embodiments, the initial impedance is in the neighborhood of 1-20 KΩ, such as about 5-15 KΩ, e.g., 1 KΩ, 2 KΩ, 3 KΩ, 4 KΩ, 5 KΩ, 6 KΩ, 7 KΩ, 8 KΩ, 9 KΩ, 10 KΩ, 11 KΩ, 12 KΩ, 13 KΩ, 14 KΩ, 15 KΩ, and 16-20 KΩ. In some embodiments, suitable impedance values after long-term use is less than 1000 KΩ, preferably less than 500 KΩ, e.g. 10-500 KΩ, 50-500 KΩ, or 50-300 KΩ. In some embodiments, long-term use is at least one week, at least one month, one to three months, one to six months, one to twelve months, one to 15 months, one to 18 months, one to 21 months, one to 24 months, or more.

The electrode composition has low input impedance over a long period of time, e.g. days, weeks, or months. In some embodiments the electrode composition possesses a low input impedance for at least two weeks, at least one month, or at least two months. In addition, the electrode composition is adhesive, maintaining good electrical contact with a subject's scalp over the same period of at least two weeks, at least one month, or at least two months. Thus, the electrode composition can be used to collect EEG signal from an animal or human subject over at least two weeks, at least one month, or at least two months.

One aspect of the invention provides an electrode comprising an electrode composition forming at least part of the electrode contact 12. In some aspects, the electrode composition comprises a polysaccharide gelling agent, a low molecular weight carbohydrate (saccharide; sugar) that is adhesive over a long period of time and retains this adhesiveness under kinetic conditions, e.g., when the subject is running, jumping, sitting, standing, sleeping, etc. The electrode also has an initial low input impedance. The input impedance of the electrode remains low over long term use, e.g., over periods of several days, weeks, or even months. In some embodiments, the electrode composition comprises: (a) a polysaccharide gelling agent; (b) a low molecular weight carbohydrate; (c) a polypeptide gelling agent; (d) a polymeric gel thickener; (e) an emulsifier; (f) a conductive carbon species; and (g) water. In some embodiments, the composition also includes one or more buffer species to maintain the pH of the composition within workable limits, e.g. pH 2 to 6, more specifically pH 2 to 4 or pH 2.9 to 3.3. In some embodiments, the buffer species is citric acid, calcium citrate, or a mixture of citric acid and calcium citrate. In some embodiments, the polysaccharide gelling agent is Methoxyl Pectin. In some embodiments, the Methoxyl Pectin is High Methoxyl Pectin. In some embodiments, the polypeptide gelling agent is gelatin. In some embodiments, the polymeric gel thickener is carboxymethyl cellulose. In some embodiments, the emulsifier is polyvinylpyrrolidone. In some embodiments, the conductive carbon species is acetylene black. In some aspects, the electrode has an initial impedance of less than about 20 KΩ, or any of the ranges of impedances recited in the previous paragraph. In some aspects, the electrode, after prolonged usage, has an impedance of less than about 300 KΩ, or any of the ranges of impedances recited in the previous paragraphs. In some aspects of the invention, the electrode composition consists only of constituents that are GRAS-compliant, i.e., they are listed by the FDA as Generally Regarded As Safe ingredients. Thus, in addition to providing low input impedance and a high degree of adhesiveness over a long period of time, the electrode composition is non-toxic and non-irritating to the scalp of the subject. Thus, the electrode composition is well adapted for long-term collection of EEG signals in the real world, outside the clinic, over a long term.

In some aspects, the electrode comprises a hydrogel polymer that is adhesive over a long period of time and retains this adhesiveness under kinetic conditions, e.g., when the subject is running, jumping, sitting, standing, sleeping, etc. The hydrogel polymer is also conductive, having an input impedance that is low, as described in the previous paragraph. The input impedance of the electrode remains low over long term use, e.g., over periods of several days, weeks, or even months, as described in the previous paragraph. In some aspects, the hydrogel comprises a co-polymer of poly(propylene glycol) diacrylate and pentaerythritol pentaerythritol tetrakis(3-mercapto-propionate) monomer. The hydrogel is capable of absorbing aqueous solutions, especially electrically conductive aqueous solutions containing solutes, such as buffered aqueous solutions. In some aspects of the invention, the aqueous solution is a physiologically acceptable aqueous buffer having a pH of about 7 to about 10. In some aspects, the poly(propylene glycol) diacrylate has an average monomer number (Mn) of about 800. In some aspects, the pentaerythritol tetrakis(3-mercaptopropionate) is about 95% pure. In some aspects, the physiologically acceptable aqueous buffer is an aqueous phosphate buffer or an aqueous bicarbonate-carbonate buffer. In some aspects, the electrode has an initial impedance of less than about 20 KΩ, or any of the ranges of impedances recited in the previous paragraph. In some aspects, the electrode, after prolonged usage, has an impedance of less than about 300 KΩ, or any of the ranges of impedances recited in the previous paragraphs.

One skilled in the art will appreciate that the precise impedance of the electrode 10 will depend, in addition to the composition described above, upon the geometry of the electrode contact 12, in that the higher the surface area of the electrode contact 12 the lower its impedance. Additionally, the impedance of the electrode contact 12 will depend upon the conductivity of the buffer. The buffer may be any physiologically acceptable buffer, meaning that it must be such that an ordinary subject, of ordinary sensitivity, will be able to tolerate its contact with the skin over long periods of time. Suitable buffers include citric acid/citrate buffers, phosphate buffers, and carbonate/bicarbonate buffers in a physiologically tolerable pH range, e.g. from 2.5 to 10. In the case of the gelatin compositions, above, the pH may range from 2 to 4, whereas the pH of the hydrogel compositions may range from about 7.2 to 9 or 7.2 to 8. The initial pH of the buffer may, once the electrode has been attached to a subject, drift over time, and the precise ending pH of the electrode contact 12 may be considerably different from its starting pH (higher or lower by 1 pH or more). Thus, as used herein, pH refers to the initial pH of the gelatin or hydrogel electrode composition.

The proximal end 14 of the electrode 10 may include a terminal jack for insertion into a receptacle on an EEG instrument, or may be permanently affixed (e.g., soldered) onto a circuit board.

The electrode 10, while especially suitable for use in an ambulatory environment, may also be employed in a less-mobile, clinical environment, where it may be considerably more convenient to avoid depilation, to affix the electrode to the subject's scalp more quickly than is permitted with conductive cement, or to keep the electrode in place for a longer period of time, such as one or more of those time periods described in previous paragraphs. While the description herein has referred to a single electrode 10 it should be apparent to those skilled in the art that plural electrodes 10 may be employed in collection of EEG signals from subjects. Due to the special features of the electrode 10 it is considered that a relatively large number of electrodes 10 may be conveniently used, especially in a clinical setting, to acquire signal for localization of seizure activity within the brain. For example, since depilation is in general not necessary with the electrode 10, plural electrodes 10 may be placed all over the scalp, and left in place for a longer period of time, without undue discomfort by the subject. Other aspects and advantages of the electrode 10 will become apparent to one skilled in the art upon consideration of the invention.

### Electrode Compositions

As noted above, an aspect of the invention is an electrode composition having low input impedance, long-lasting and durable adhesiveness, and low toxicity. In some embodiments, the electrode composition comprises: (a) a polysaccharide gelling agent; (b) a low molecular weight carbohydrate; (c) a polypeptide gelling agent; (d) a polymeric gel thickener; (e) an emulsifier; (f) a conductive carbon species; and (g) water.

A polysaccharide gelling agent can include any polysaccharide that has a tendency to form a gel at standard temperature and pressure. Pectins are considered especially desirable in this regard, as they are Generally Regarded As Safe (GRAS), and perform well under the required conditions. In some embodiments, the pectin is advantageously a methoxyl pectin, such as high methoxy pectin, although low methoxy pectin may be used as well. Other suitable polysaccharide gelling agents include carrageenan, agar, algin, gellan gum, psyllium and chitin.

The low molecular weight carbohydrate may be any mono- or di-saccharide carbohydrate, such as sucrose, maltose, or lactose.

A polypeptide gelling agent can include any polypeptide that has a tendency to form a gel under standard temperature and pressure. Gelatins are considered especially desirable in this regard, as they are GRAS, and perform well under the required conditions, especially in combination with pectins such as methoxyl pectin, and especially high methoxyl pectin.

Polymeric gel thickeners may be used to improve the consistency, long-term hydration and long-term stability of the electrode composition. Suitable polymeric gel thickeners include soluble celluloses, such as carboxymethyl cellulose (CMC). Other suitable polymeric gel thickeners include guar gum, xanthan gum, and locust bean gum.

An emulsifier may be used to assist in mixing of the various ingredients, dispersing the conductive carbon species in the gel, and generally improve the homogeneity of the composition. A suitable emulsifier is polyvinylpyrrolidone (PVP), though other emulsifiers may be used, such as other food safe or GRAS nonionic surfactants. Other suitable emulsifiers include gum arabic, and lecithin.

A conductive carbon species is provided to improve the long-term conductivity of the composition. A suitable conductive carbon species is acetylene black. However, other conductive carbon species, such as graphite or nanotubes may be employed.

In some embodiments, the composition also includes one or more buffer species to maintain the pH of the composition within workable limits, e.g. pH 2 to 6, more specifically pH 2 to 4 or pH 2.9 to 3.3. In some embodiments, the buffer species is citric acid, calcium citrate, or a mixture of citric acid and calcium citrate. Citric acid may also provide some preservative value to the composition. The counter ion to citrate in the citrate-based buffer may be any suitable counter ion, including sodium, potassium, magnesium, etc. Other suitable species may include phosphoric acid or phosphate buffer species, boric acid or borate buffer species, sulfuric acid or sulfate buffer species, etc.

The composition may also comprise other ingredients, such as antioxidants, preservatives, protectants, antimicrobials, antifungals, etc.

### EEG Signal Processing

As noted above, an aspect of the invention is detection of seizures in real time and rapid alerting of responsible personnel of seizures when they happen (not minutes or hours later). This invention overcomes the problem of handling large data sets, most of which comprise EEG signals indicative of interictal activity (normal brain activity), to rapidly identify the relatively small proportion of EEG signals indicative of ictal events. In some embodiments, the invention provides a preliminary signal indicative of a probable seizure, and then confirms or refutes the preliminary signal. In some embodiments, the preliminary signal may be a yellow light on a device wearable by the subject or a responsible person, or some suitable indication in a mobile device. In some embodiments, the confirmatory signal may be a red light on a device wearable by the subject or a responsible person, or some suitable indication in a mobile device. In some embodiments, if no seizure is detected, or if a preliminary signal (e.g., yellow light) is refuted, a green light on a device wearable by the subject or a responsible person, or some other suitable indication in a mobile device. Such other suitable indications can include text messages, optionally with accompanying audible indicators. Responsible persons can include, in the case of humans, caregivers, physicians, custodians, the patient himself or herself, and in the case of animals, such as pets, veterinarians, caregivers, owners, and other companions.

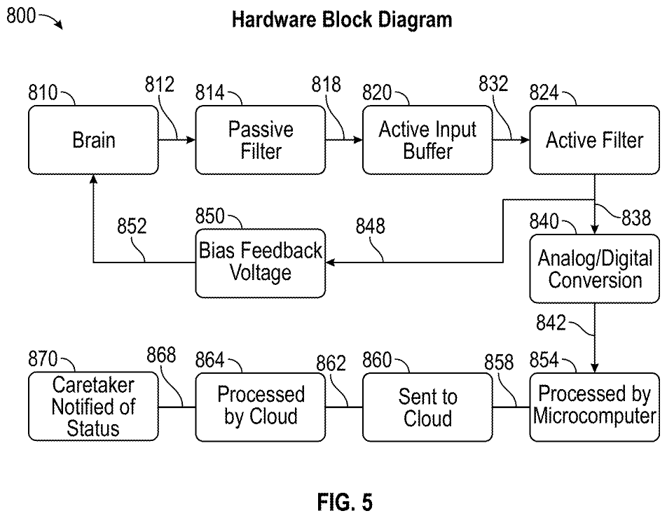

Processing of an EEG signal collected from an ambulatory subject may be carried out as depicted in block diagram 800 in FIG. 5. An analog voltage signal 812 is collected from the brain 810 of a subject. The analog voltage signal 812 may be collected using an electrode 10, as described herein, attached to the subject's scalp using, e.g., an adhesive, low input impedance, non-toxic composition as described herein. The analog voltage signal 812 is then passed through a passive filter 814 to provide a passively filtered signal 818, which is passed to an active input buffer 820. The active input buffer 820 then passes the buffer output signal 832 to an active filter 824, which then passes the actively filtered signal 838 to an analog to digital converter (ADC) 840. The active filter 824 provides a bias signal 848 which detects the common mode voltage of the electrodes 848 through a bias negative feedback voltage loop 850, which feeds an inverted common-mode signal 852 back to the brain 810, e.g., through a bias electrode 10, as described herein, thereby providing an electrical common for the signal and restricting the common-mode movement to a narrow range. The ADC 840 converts the analog signal 838 to provide a digital signal 842, which is provided to a microcomputer 854. The passive filter 814, active input buffer 820, active filter 824, and ADC 840 may all be located on a single circuit board with a power supply (not shown), and a transmitter, such as a WIFI, Bluetooth, or other short-range transmitter adapted to pass the digital signal 842 to the microcomputer 854. The microcomputer 854 receives the digital signal 842 from the ADC 840, optionally processes the digital signal 842, e.g. by applying a suitable CODEC, and then passes a processed signal 858, e.g., over a wireless network or the Internet, to a cloud server 860, which processes the digital cloud signal 862, e.g. by transforming the signal from the time domain into a special domain, graphing the signal, and processing the graphed signal through cross-correlation and/or machine deep learning to detect ictal events. Such processing is indicated as “Processed by the Cloud” 864. Once an ictal event is detected, a warning signal 868 is sent via a suitable communication device 870, such as a personal computer, cell phone app, tablet app, or similar communication means, to notify a caretaker of the ictal event.

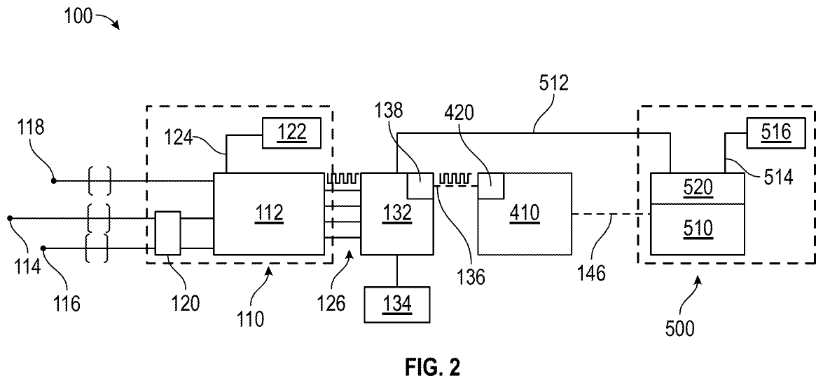

A system according to the invention is presented in FIG. 2, where system 100 is a representative system of the invention. Analog signal collection from the subject is effected via an EEG signal collector 110, which comprises an analog front-end chip 112, which receives power from power supply 122. Typically, an analog front-end chip 112 will require both analog and digital potentials which are represented by power input 124. The analog front-end chip 112 provides a bias potential through bias electrode 118. Scalp potentials representative of brain waves are collected by electrodes 114 and 116 and filtered by passive filters 120, through which they pass to the analog front-end chip 112. The analog front-end chip 112 converts the analog input signal to a digital output signal, which is passed through digital output leads 126 to a digital interface 132. In a suitable analog front-end chip 112, input buffering, differential (active) filtration, amplification, and analog-digital conversion are combined in a single chip, thereby simplifying device design. Suitably, at least the signal collector 110, comprising at least the passive buffer 120, the front-end chip 112, and the power supply 122, is incorporated into a wearable device, such as a hat, collar, belt, necklace, headband, or similar wearable device. The wearable device may be adapted to fit in close proximity to a subject's head, thereby permitting the use of short leads for the electrodes 114, 116, and 118, which reduces noise induction and signal loss in the leads. Shorter leads may also result in reduced physical stress on the electrodes 114, 116, and 118.

The digital interface 132 receives the digital signal from digital outputs 126 and presents the digital signal to an analyzer 410 through connection 136. The digital interface 136 may include (but does not have to include) a means for digital transmission by way of a network to the signal analyzer 410. For instance, the digital interface 132 may include a digital transceiver 138, which sends the digital signal over the Internet or similar network to a digital transceiver 420 local to the signal analyzer 410, which transmits the digital signal to the signal analyzer 410.

The digital analyzer 410 analyzes the digital signal to detect likely ictal events and alert a responsible person through an alert subsystem 500 via connection 146. The alert subsystem 500 includes an alert subroutine 510, which may send an intermediate alert signal by alert feedback circuit 512 to the digital interface 132, which in turn actuates an intermediate alert signal (e.g., a yellow light, an LCD indicating “Potential Seizure,” or both, or some other indicator of a potential seizure) on alert indicator 134 connected to the digital interface 132. (The default value of the alert indicator 134 is normal, or no seizure, which may be indicated in various ways, e.g. by lighting of a green light, an LCD display of the word “NORMAL,” both, or some other normal indicator.) Although the alert feedback circuit 512 is depicted as a connection between the alert subsystem 500 and the digital interface 132, this is done only to show signal flow. In actual practice, the alert feedback circuit 512 would comprise a transceiver 520 adapted to transmit the alert feedback signal to the digital transceiver 138 of the digital interface 132. The alert feedback system may include, in addition the transceivers, 138 and 520, additional circuitry, such as provided by the Internet, a local area network (LAN), and/or other network system. The alert subroutine 510 may alternatively, or in addition, activate an alert device 516 through alert channel 514. The alert device 516 may be a wearable device, a computer, or a handheld device, such as a cellular phone or web-enabled tablet, which may produce an alert signal, such as an audible or visual signal (or both), to a responsible person that a seizure event is possible. Either or both of alert circuitry 512 or alert channel 514 may be effected in whole or in part over a network, such as a wireless network, the Internet, or some other network, or combination of two or more of the foregoing. The responsible person may be a caretaker, such as a physician, physician's assistant, LPN, RN, family member, or other caretaker, may, upon receiving an alert that a seizure is in progress, administer one or more treatments to the subject. Such treatments may include administration of an acute anti-seizure medication (such as rectal or nasal diazepam or lorazepam), administration of a dose of the subject's current anti-seizure medication (e.g., if a dose was inadvertently missed), administration of a higher dose of the subject's current anti-seizure medication, administration of a different anti-seizure medication. The caretaker may also alert a secondary caretaker of the seizure event. For example, a human pet owner who receives an initial alert may contact a veterinarian to receive instructions from the veterinarian on treatment of the subject (pet). Alternatively, a veterinarian who receives the initial alert may contact a pet's (subject's) owner or custodian with instructions for the caretaker to initiate, change, or augment treatment. Likewise, a family member of a human subject who receives the initial alert may contact the subject's physician to receive treatment instructions; or, a physician who receives the initial alert may contact the subject's primary caretaker to provide instructions for treatment by the caretaker. Once treatment instructions are received by the caretaker from a veterinarian, physician or other competent healthcare professional, the caretaker may then treat the subject accordingly, e.g. with one of the treatment options outlined above, or may arrange for transport of the subject to a suitable facility for the subject to receive such treatment.

In some embodiments, the whole of EEG signal collector 110 and at least part of digital interface 132 are included in a wearable device to be worn by the subject. For example, EEG signal collector 110 and a digital transceiver 138, which are part of digital interface 132, may be incorporated into a single wearable device, such as a hat. (They may also be separated into two connected wearable devices, such as a headband and a necklace, or a soft helmet and a backpack, etc.) In such case, a second transceiver, also part of digital interface 132 receives the digital signal and presents it to the digital analyzer 410, e.g., by Bluetooth®, WIFI, or other radio-frequency data transfer protocol, for analysis.

In some embodiments the EEG signal collector 110 and the digital interface 132 may be located on a common circuit board, which may also include the digital transceiver 138 and/or the alert indicator 134.

The digital analyzer 410 may incorporate software that both detects suspected ictal events, and then performs additional analysis to confirm or refute the initial indication of a potential seizure. In the case that additional analysis (confirmation analysis) is performed and the initial indication of seizure is confirmed, the digital analyzer 410 may signal the alert subsystem 500, which, employing alert subroutine 510 and transceiver 520, may send a confirmatory alert signal by alert feedback circuitry 512 to the digital interface 132, which in turn may actuate a seizure confirmed alert signal (e.g., a red light) on alert indicator 134 connected to the digital interface 132. The alert subroutine 510 may alternatively, or in addition, activate alert device 516 through alert channel 514, thereby informing a responsible person (as described herein) that the seizure status has been confirmed. In the case that additional analysis (confirmation analysis) is performed and the initial indication of seizure is refuted, the digital analyzer 410 may signal the alert subsystem 500, which, employing alert subroutine 510, may send a normal alert signal by alert feedback circuitry 512 to the digital interface 132, which in turn may actuate a normal alert signal (e.g., a green light) on alert indicator 134 connected to the digital interface 132. The alert subroutine 510 may alternatively, or in addition, activate alert device 516 through alert channel 514, thereby informing a responsible person (as described herein) that the seizure status has been refuted. The alert device 516 may be a wearable device, a computer, or a handheld device, such as a cellular phone or web-enabled tablet, which may produce an alert signal, such as an audible or visual signal, to a responsible person that a seizure event is possible. As noted above, either or both of alert circuitry 512 or alert channel 514 may be effected, in whole or in part over a network, such as a wireless network, the Internet, or some other network, or combination of two or more of the foregoing.

The system may include other features, such a system reset channel to reset the alert status to its default (normal) upon signaling by subject or a responsible person, or after passage of a preselected period of time.

The operation of the digital analyzer 410 may be understood in terms of the algorithm 700 set forth in FIG. 4. In step S710 a digitized biopotential collected from a subject is recorded. In step S720 transforms are applied to map the time sequence data into a visual graph space. In step S730 the transformed data are plotted in a graph. In step S740, the graphic data are fed into a convolutional neural network, which is programmed to detect and discriminate ictal from interictal events. In step S750 the neural network classifies the recorded biopotential as ictal (seizure) or interictal (non-seizure). If a seizure is detected, a signal may be sent to a physician or caretaker, notifying the physician or caretaker of the seizure, whereby the physician or caretaker may take action, such as administering acute anti-seizure medication, increasing dosage of existing medication, changing the subject's seizure medication, or changing the subject's seizure therapy.

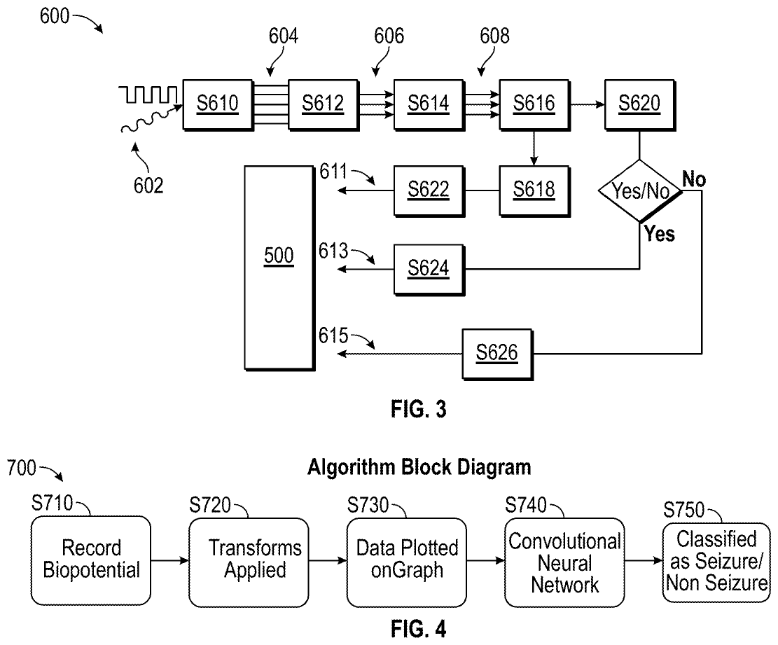

The operation of the digital analyzer 410 may be further understood upon consideration of the block diagram 600 set forth in FIG. 3. The digital signal 602 is received, as described above, e.g. from digital interface 132 (FIG. 2). In step S610 the digital signal is segmented into time sequences 604 of various lengths (e.g., 10 s, 20 s, 30 s, 40 s, 50 s, 60 s). Alternatively, the time sequences may all be of a single length (e.g., a suitable length sequence in a range of 1 s to 240 s). The length of the time sequence may be chosen to optimize the balance between tractability of the size of the data set, quickness of seizure detection, and quality of seizure detection. For example, a large data set (longer time series) may provide more definite seizure detection, but may sacrifice speed of an initial alert in the balance. On the contrary, a short time series may ensure fast processing and alerting, but may sacrifice signal quality and accuracy. One skilled in the art will appreciate that the quality of the device will depend on optimizing these parameters.

At least initially, while the system is in learning mode, time series of different lengths may be prepared by step S610, permitting evaluation and optimization of the time series lengths. Once the time series 604 are prepared, they are passed to step S612 where they are transformed into at least one different domain. For example, transforms that may be performed include Fourier Transform (FFT), Power Band, and Wavelet transform. These transforms 606 are then passed to step S614, where they are physically plotted into graphs 608. These physical graphs may include electronic graphs recorded in physical memory in S616. These physical graphs are then passed to cross correlation step S618 and machine deep learning step S620. In cross correlation step S618, cross correlation is performed on the physical graphs, e.g. using a graphics processing unit (GPU) to detect a potential ictal signal. On determination that a graph represents a potential ictal signal, cross correlation step S618 activates potential seizure alert step S622, which sends a potential seizure alert signal 611 to alert subsystem 500, whose operation is described above.

In machine deep learning step S620, machine deep learning techniques are performed on the physical graphs, e.g. using a graphics processing unit (GPU), to detect an ictal signal, which may be considered a “confirmed” ictal (or interictal) signal. In some embodiments, convolutional neural networks, recurrent neural networks, or both, are used in a machine deep learning model to determine whether a time series (or more properly a graphical representation of a transform of a time series) represents an ictal event. If it is confirmed that a graph represents an ictal signal (Y in the Y/N decision node), machine deep learning step S620, through the Y logic arm, activates confirmed seizure alert step S624, which sends a confirmation seizure alert signal 613 to alert subsystem 500, whereby alert indicator 134 is set to status “Seizure Confirmed,” which may be indicated by a red light (which may blink), an LCD display indicating “Seizure Confirmed, or both, or some other indicator that a seizure status has been confirmed. A corresponding signal 514 may also be sent to one or more responsible persons through an alert device 516. If, on the other hand, it is determined that the signal does not represent an ictal signal (N in the Y/N decision node), machine deep learning step S620, through the N logic arm, activates normal condition alert step S626, which sends normal condition signal 615 to alert subsystem 500, whereby the alert indicator 134 is set to its default (normal) status, indicating no seizure. This status may be indicated in various ways, e.g. by lighting of a green light, an LCD display of the word “NORMAL,” both, or some other normal indicator. A corresponding signal 514 may also be sent to one or more responsible persons through an alert device 516.

### Detection and Treatment

Provided herein are methods of alerting a caretaker (e.g., a doctor, nurse, family member, etc.) that a subject is suffering from a seizure. Such alerts may be delivered to the caretaker via an electronic means, such as a text message or notification via a dedicated application (app) on a smart phone, tablet, or other Internet-connected device. The alert may also include a visual indication on the wearable device on the subject's body, such as a lighted indicator, as described herein. The lighted indicator may indicate a potential ictal event, and, when optionally confirmed as described herein, may also indicate an actual ictal event. The purpose of the alert is to give real-time information to the caretaker that the subject is in need of attention, such as anti-seizure treatment.

In some embodiments, there is provided a method of detecting and alerting a caretaker of an ictal event (seizure) in a subject, said system comprising: (a) collecting an analog voltage signal 812 from the scalp of a subject; (b) applying at least part of the analog signal to an analog to digital converter (ADC) 840 to provide a digital output signal 842; (c) passing a digital output signal 842 to a cloud server 860; (d) transforming and graphing the digital output signal 842 to a digital graph space and graphing the digital graph space signal; (e) applying the digital graph space signal to a neural network to detect an ictal event; and (f) when an ictal event is detected, sending an alert to a caretaker.

In some embodiments, there is provided a method of detecting and alerting a caretaker of an ictal event (seizure) in a subject, said system comprising: (a) collecting an analog voltage signal 812 from the scalp of a subject; (b) filtering the analog voltage signal 812 to provide a filtered analog signal; (c) applying the filtered analog signal to an analog to digital converter (ADC) 840 to provide a digital output signal 842; (d) passing a digital output signal 842 to a cloud server 860; (e) transforming and graphing the digital output signal 842 to a digital graph space and graphing the digital graph space signal; (f) applying the digital graph space signal to a neural network to detect an ictal event; and (g) sending an alert to a caretaker when an ictal even is detected.

Also provided herein are methods of treating a subject. The methods include, when an ictal event is detected, administering to the subject an anti-seizure therapy. Such anti-seizure therapies may include fast-acting anti-seizure medications, such as rectal gels or intranasal sprays. Other anti-seizure therapies may include increasing a dosage, frequency, or both, of an existing pharmaceutical therapy. Other anti-seizure therapies may include a change of therapeutic agent.

In some embodiments, there is provided a method of treating a subject having a seizure disorder, comprising: (a) collecting an analog voltage signal 812 from the scalp of a subject; (b) applying at least part of the analog signal to an analog to digital converter (ADC) 840 to provide a digital output signal 842; (c) passing a digital output signal 842 to a cloud server 860; (d) transforming and graphing the digital output signal 842 to a digital graph space and graphing the digital graph space signal; (e) applying the digital graph space signal to a neural network to detect an ictal event; and (f) when an ictal event is detected, administering an anti-seizure therapy to the subject.

In some embodiments, there is provided a method of treating a subject having a seizure disorder, comprising: (a) collecting an analog voltage signal 812 from the scalp of a subject; (b) filtering the analog voltage signal 812 to provide a filtered analog signal; (c) applying the filtered analog signal to an analog to digital converter (ADC) 840 to provide a digital output signal 842; (d) passing a digital output signal 842 to a cloud server 860; (e) transforming and graphing the digital output signal 842 to a digital graph space and graphing the digital graph space signal; (f) applying the digital graph space signal to a neural network to detect an ictal event; and (g) when an ictal event is detected, administering an anti-seizure therapy to the subject.

### Example 1: Gelatin Electrode Composition

An exemplary electrode composition according to the present invention is prepared as follows:

Powder Preparation:

Weigh each of the following components: high methoxyl pectin, gelatin, carboxymethyl cellulose, and citric acid, with the high methoxyl pectin separated from the other powders.

Mix gelatin, carboxymethyl cellulose, and citric acid together thoroughly.

In a separate container, measure out low molecular weight carbohydrate.

Carbon Solution Preparation:

Add H₂O to a new container.

Carefully weigh the polyvinylpyrrolidone and acetylene black powders, then add both to the beaker.

Immediately begin stirring the solution.

Sonicate solution for 600 seconds, or until the carbon powder is fully dispersed.

Electrode Solution Mixing and Heating:

Heat the carbon dispersion to 60° C. using a hot water bath or double boiler.

Slowly add the high methoxyl pectin powder mixture to the water while stirring.

Next, slowly add the mixed gelatin, carboxymethyl cellulose, and citric acid powders while stirring and stir until dissolved.

Next, slowly add the low molecular weight carbohydrate, and stir until dissolved.

Once the mixture is completely dissolved, remove it from the heat source.

Continue stirring for 5 minutes, or until the mixture begins to thicken.

Curing Process:

Allow the mixture to set for at least 8 hours.

The resulting composition possesses an initial pH of 2.9 to 3.3, and an initial impedance of 5-250 kΩ. After 3 months of storage, the input impedance has increased to 150-400 kΩ

The reference numbers recited in the below claims are solely for ease of examination of this patent application, and are exemplary, and are not intended in any way to limit the scope of the claims to the particular features having the corresponding reference numbers in the drawings.

1. An electrode composition comprising:
a. a polysaccharide gelling agent;
b. a low molecular weight carbohydrate;
c. a polypeptide gelling agent;
d. a polymeric gel thickener;
e. an emulsifier;
f. a conductive carbon species; and
g. water.

2. The electrode composition of claim 1, wherein the polysaccharide gelling agent is Methoxyl Pectin.

3. The electrode composition of claim 1, wherein the Methoxyl Pectin is High Methoxyl Pectin.

4. The electrode composition of claim 1, in which the polypeptide gelling agent is gelatin.

5. The electrode composition of claim 1, in which the polymeric gel thickener is carboxymethyl cellulose.

6. The electrode composition of claim 1, in which the emulsifier is polyvinylpyrrolidone.

7. The electrode composition of claim 1, in which the conductive carbon species is acetylene black.

8. The electrode composition of claim 1, additionally comprising a buffer species.

9. The electrode composition of claim 1, wherein the buffer species comprises citric acid, calcium citrate, or both.

10. The electrode composition of claim 1, having a pH of from about 2 to about 4.

11. A process of preparing an electrode composition of claim 1, comprising:
a. placing and mixing the polypeptide gelling agent, the polymeric gel thickener, and optionally a buffer powder in a first container;
b. charging the water, the emulsifier, and the conductive carbon species powder into a second container;
c. thoroughly dispersing the conductive carbon species powder in the water to form a dispersion;
d. heating the dispersion in the second container to about 60° C.;
e. introducing the polysaccharide gelling agent into the second container and stirring to dissolve the polysaccharide;
f. charging the mixed polypeptide gelling agent, polymeric gel thickener, and buffer powder from the first container into the second container and stirring until the charged powder is dissolved;
g. charging the low molecular weight carbohydrate into the second container and stirring until the low molecular weight carbohydrate is dissolved;
h. discontinuing heating of the dispersion in the second container and stirring until the dispersion begins to thicken; and
i. allowing the dispersion to set to form the electrode composition.

12. A method of detecting and alerting a caretaker of an ictal event (seizure) in a subject, said system comprising:
a. collecting an analog voltage signal 812 from the scalp of a subject,
b. applying at least part of the analog signal to an analog to digital converter (ADC) 840 to provide a digital output signal 842;
c. passing a digital output signal 842 to a cloud server 860;
d. transforming and graphing the digital output signal 842 to a digital graph space and graphing the digital graph space signal;
e. applying the digital graph space signal to a neural network to detect an ictal event; and
f. when an ictal event is detected, sending an alert to a caretaker.

13. (canceled)

14. A method of detecting and alerting a caretaker of an ictal event (seizure) in a subject, said system comprising:
a. collecting an analog voltage signal 812 from the scalp of a subject,
b. filtering the analog voltage signal 812 to provide a filtered analog signal;
c. applying the filtered analog signal to an analog to digital converter (ADC) 840 to provide a digital output signal 842;
d. passing a digital output signal 842 to a cloud server 860;
e. transforming and graphing the digital output signal 842 to a digital graph space and graphing the digital graph space signal;
f. applying the digital graph space signal to a neural network to detect an ictal event; and
g. sending an alert to a caretaker when an ictal even is detected.

15.-20. (canceled)
